(19) United States
(12) Patent Application Publication
(10) Pub. No.: US 2001/0008764 A1
(43) Pub. Date: Jul. 19, 2001

(54) FERTILITY-RELATED PROTEINS, AND METHODS OF USE THEREOF

(76) Inventors: ROY AX, TUCSON, AZ (US); MARY BELLIN, TUCSON, AZ (US); TOD MCCAULEY, TUCSON, AZ (US); ZHANG HUANMIN, TUCSON, AZ (US)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/055,671

(22) Filed: Apr. 7, 1998

Related U.S. Application Data
(63) Continuation-in-part of application No. 08/487,168, filed on Jun. 7, 1995, now Pat. No. 5,962,241.
Application No. 08/487,168 is a continuation-in-part of application No. 07/386,954, filed on Jul. 28, 1989, now abandoned.

(51) Int. Cl. A61K 39/395; C07K 16/00; C12P 21/08

(52) U.S. Cl. 435/7.1; 424/143.1; 424/141.1; 424/130.1; 530/388.22; 530/388.1

(57) ABSTRACT

Two new heparin-binding proteins (HBP) in bovine seminal fluid are characterized. Both proteins are strongly correlated with enhanced fertility. The first protein exhibits a molecular mass of 31-kDa in SDS-PAGE electrophoresis, and has an N-terminal sequence, as well as internal sequences, closely correlated with deoxyribonuclease I-like protein. The second protein is identified as a tissue inhibitor of metalloproteinases, and has a 24-kDa molecular mass in SDS-PAGE electrophoresis under reducing conditions, and a 21.5 kDa under non-reducing conditions. N-terminal sequence information for both proteins is provided. Assays for these proteins may be employed to indicate bulls of higher fertility.

### RELATED APPLICATIONS

This application is related to and a Continuation-In-Part of U.S. patent application Ser. No. 08/487,168 filed Jun. 7, 1995, the entire content of which is incorporated herein by reference. That application is in turn a CIP application of U.S. Pat. No. 5,434,139, itself a continuation of U.S. patent application Ser. No. 07/386,954 filed Jul. 28, 1989. The entire content of U.S. Pat. No. 5,434,139 is incorporated herein by reference.

### BACKGROUND OF THE INVENTION

1. Field of the Invention

This invention pertains to proteins, detected by monoclonal antibody assay, found in seminal fluid that are indicated to be directly related to fertility in mammals. This invention pertains to methods of predicting which candidates within a male mammalian population are likely to be the most fertile breeders, by assaying for the newly identified proteins.

2. Background of the Prior Art

Reproductive capacity has a tremendous impact on the animal industry. Reproductive merit is five times more important, economically, than growth performance, and at least ten times more important than product quality, as applied to beef cow/calf producers. Trenkle and Wilham, 1977. Selection of livestock males with the highest fertility potential is one way of improving reproductive efficiency, and offsetting potentially large economic losses that are incurred as a result of poor fertility. Regrettably, criteria to accurately select males exhibiting the highest fertility are poorly defined, and the physiological importance of parameters that are reliable indicators of fertility potential is poorly understood. Morphological examination of sperm cells and semen, alone, is insufficient to predict male reproductive success. Taking even single sire situations, and assessing outcomes from artificial insemination, bulls with acceptable semen characteristics by currently accepted evaluations vary widely in their ability to impregnate cows. Variations in fertility between bulls that produce similarly assessed semen contributes greatly to the problem of identifying bulls with the highest fertility potential.

Among the characteristics commonly evaluated in an attempt to predict fertility are included several characteristics of sperm viability. Measurements of motility, acrosomal integrity, cervical mucus penetration and cellular content of DNA, enzymes and lipids are all characteristics that form the general basis for sperm cell viability, and have been studied under different conditions. Saacke, 1983. Unfortunately, correlation of results obtained from this type of analysis and fertility is not reliable. Criteria such as motility, once thought to be highly correlated to fertility, now appear to be less important. This can be explained by a variety of factors, including inaccurate motility characterization, inaccurate measurements of fertility, influence of non-semen factors and perhaps most importantly, effects of other measures on fertility. O'Connor et al., 1981.

Non-return rate is an estimate of relative fertility, calculated by commercial artificial insemination organizations, expressed as a percentage of cows not re-bred to the same bull within 30-60 days post-insemination. It is a crude but widely employed reflection of a bull's ability to impregnate cows. The cellular trait most highly correlated with non-return rate is the percentage of intact acrosomes. Saacke, 1984.

Another standard test for assessing fertilization capacity, particularly in range beef bulls, is the Breeding Soundness Exam or BSE. This involves determination of scrotal circumference, sperm motility, sperm morphology and provides a classification of bulls as satisfactory potential breeders if they equal or exceed minimum thresholds. Chenoweth et al., 1992. While BSE is suited for detecting bulls of questionable fertility, it is inadequate to predict differences in fertility among sound breeding bulls that pass the BSE. For example, two bulls previously accepted as sound breeding sires according to BSE results may vary widely in fertility. Bellin et al., 1994, 1996, 1998. The protocols for determining individual elements of the BSE are also tedious, and the BSE is not widely used as a part of bull management techniques when large cross-sections of beef operations are evaluated.

Accordingly, although sperm viability is one requisite trait for determining fertilizing capability, it alone, is not sufficient to identify highly fertile potential breeders. Among sources considered are biochemical assessment of sperm surface components. Graham et al., 1990. An inconsistent relationship between sperm viability characteristics commonly evaluated in semen analysis and fertility has been demonstrated, however, and accordingly, other factors contributing to fertility have been studied. These include comparing acrosome reaction in response to treatment with heparin-like glycosaminoglycans (GAG). Lenz et al., 1988 and Whitefield et al., 1992. These studies have strongly suggested that factors involved in capacitation of mammalian sperm, and in particular, the acrosomal reaction, may be a key to indicating the potential for high fertility. Capacitation itself is a difficult process to study. Failure to acrosome react, for example, is not clearly indicative of incapacitated sperm, even though sperm are typically considered to have undergone capacitation if they acrosome react in response to a specific stimulus. It is generally accepted that capacitation involves substantial modification of sperm surface glyconjugates. Saling, 1989. Alterations of surface components during sperm capacitation are believed to be a pre-requisite to successful fertilization. Competing events involved in capacitation and other pre-fertilization events have led those of skill in the art to identify fertility markers in seminal fluid, excluding the results of abnormal semen characteristics. Four proteins in bull seminal plasma that were positively correlated with bull fertility were identified by Killian et al., 1993. Two-dimensional gel electrophoresis of seminal plasma from bulls differing in fertility by a relatively narrow range indicated that relative density of two proteins (26 kDa, pI 6.2 and 55 kDa, pI 4.5) predominated in highly fertility bulls. Two other proteins were more predominant in seminal plasma from lower fertility bulls. The 55 kDa protein was identified as Osteopontin, Cancel et al., 1997, and the 26 kDa species was identified as Prostaglandin D Synthase (Gerena et al., 1998). Similar analysis of bovine seminal plasma revealed three size classes of heparin and binding protein of 15-17, 24 and 31 kDa that bound to epididymal sperm in vitro. Miller, et al. 1990. The heparin binding proteins in bovine seminal fluid are likely linked to bull fertility. The 31 kDa heparin binding protein (HBP) was indicated to be correlated with bull fertility potential. Bellin et al., 1994 A monoclonal antibody (M1) was generated against high affinity HBP purified from seminal fluid. Bellin et al., 1996. The entire content of this disclosure is incorporated herein by reference.

None of the indicators of potential fertility developed to date are totally satisfactory as a reliable, laboratory method of indicating fertility potential, and recognizing the enormous economic gains that are available by selecting, for breeding, males with potential higher fertility. This is a significant economic issue, as, for every 1,000,000 animals weaned in the United States, a decrease of reproductive efficiency by just one percentage point is equivalent to an economic loss of $4.5 million. In particular, it remains an object of the industry to provide proteins, which, either alone, or considered as a battery, are adequate screening elements to identify males likely to be higher in fertility.

### SUMMARY OF THE INVENTION

Applicant's invention meets the above-identified objects, and others made more abundantly clear by the disclosure set forth below, in the identification of two novel antigens highly correlated with potential male fertility. A first HBP, a 31-kDa fertility-associated antigen (FAA) was isolated by heparin-affinity chromatography and reversed-phase high performance liquid chromatography to near homogeneity. The N-terminal sequence analysis of pure FAA indicates it to be closely related to a recently identified deoxyribonuclease (DNase) I-like protein. Internal amino acid sequences generated from lys-C digested FAA bore substantial homogeneity to sequences from the same DNase I-like protein. FAA is not glycosolated, and is indicated to be a basic peptide with pI of approximately 8.1. The protein is detected in at least the seminal vesicles and possibly the bulbourethral gland homogenates. FAA extracted from sperm membranes by treatment with hypertonic media was identical to seminal fluid-derived FAA, as determined by electrophoretic mobility and Western Blotting assays.

A second HBP, a 24-kD-protein isolated from Bovine Seminal Fluid, has also been highly correlated with fertility. The N-terminal amino acid sequence and the partial cDNA sequence corresponding to this protein identify it as a member of the family of tissue inhibitors of metalloproteinase (TIMP). The 24-kDa protein corresponds in size to a protein previously recognized in Western blots of sperm extracts probed with a monoclonal antibody binding to HPB and identified as HBP-21.5. Bellin et al., 1996, 1998. The mobility in SDS-PAGE for this particular protein was greater for the unreduced than the reduced form. The band migrated at approximately 24-kDa when run in the presence of 2-mercaptoethanol, and migrated at approximately 21.5-kDa under non-reducing conditions. The 21-kDa protein shares significant homology with a 21-kDa protein purified from human melanoma cells, and closely reassembles a Bovine Metalloproteinase Inhibitor purified from aortic endothelial cells identified as TIMP-2. DeClerck et al., 1989 and Boone et al., 1990.

### BRIEF DESCRIPTION OF THE DRAWINGS

FIG. 1 gives the N-terminal sequence, as well as two internal amino acid sequences, for FAA, and shows their alignment with corresponding sequences of DNAS1L3 (GenBank Accession No. U56814).

FIG. 2 provides the N-terminal amino acid sequence from HBP 24 as well as its alignment with the corresponding sequences for a Bovine Metalloproteinase Inhibitor and human TIMP-2.

### DETAILED DESCRIPTION OF THE INVENTION

The newly characterized and identified fertility-associated proteins of this invention were initially identified by immunoassay with a monoclonal antibody expressed by a hybridoma cell line S1A5C8G10H12, the monoclonal antibody being designated as M1, which has been deposited at the ATCC, 10801 University Boulevard, Manassas, Va. 20110 under Accession No: HB11915. A similar hybridoma, expressing a related monoclonal antibody M2 was deposited under Accession No: HB11916. These deposits were made on Jun. 2, 1995. Monoclonal antibody M1 recognizes HBP which coat the acrosomal and post-acrosomal regions of ejaculated sperm. These proteins are absent from the surface of epididymal sperm. Immunoblots of solubilized sperm proteins probed with M1 detected HBP variance of approximately 31 and 24 kDa. These proteins have been identified as FAA (31 kDa) and TIMP-2 (24 kDa). These proteins were isolated from bull semen. As discussed herein below, however, these proteins demonstrate a similarity that cuts across mammalian species, and thus while a preferred embodiment involves the assessment of bull semen for fertility, similar assessments may be made of semen of other mammalian species, including humans. For clarity of discussion, these two proteins are discussed separately, below, but are bound by the same monoclonal antibody M1, and are both characterized as fertility-associated proteins.

### Fertility-Associated Antigen

A fertility-associated antigen (FAA) is strongly correlated with higher fertility bulls. This is reflected in Table 1.

TABLE 1
Relationship of fertility-associated antigen 9 FAA) on sperm and
pregnancy outcome of bred cows.¹

| | |
|---|---|
| Method of detection | Western blot |

| | | |
|---|---|---|
| Fertility-associated antigen (FAA) | Present | Absent |
| Number of cows pregnant/total palpated | 3469/4105 | 578/819 |
| Fertility (%) | 84.5 | 70.6 |

¹FAA was detected on sperm by Western blotting of whole sperm extracts. Bulls (2-18 per group) were pastured for 60d at a ratio of 1 bull:25 cows after segregating on the basis of FAA analysis. Pregnancies were determined by palpation per rectum after (an additional) 60d post-breeding season.

Purified FAA was obtained according to the methods set forth herein below, and transferred to a PVDF membrane, and analyzed for N-terminal amino acid sequence. This gave a 26 amino acid sequence LKIXSFNVRSFGESKKAGFNAMRVIV that had a substantial (73%) homology to that of a recently identified deoxyribonuclease I-like protein (DNAS1L3) Rodriguez et al., 1997.

Two internal amino acid sequences were generated from lys-C digested FAA. These sequences were 85% and 90% identical to corresponding sequences of the same DNAS protein. FAA is not glycosolated, and is indicated to be a basic peptide with a pI of approximately 8.1. FAA was detected in homogenates of seminal vesicles in bulbourethral glands. FAA extracted from sperm membranes by treatment with hypertonic media was identical to seminal fluid-derived FAA.

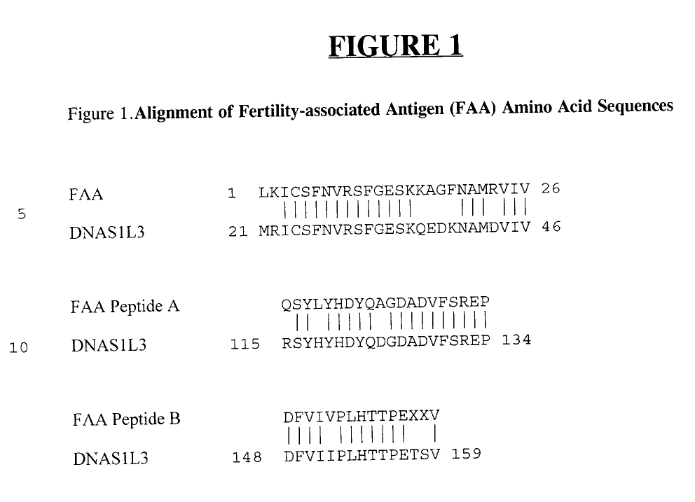

Purification of FAA to near homogeneity using heparin affinity and reversed-phase high performance liquid chromatography (RP-HPLC) gave 20 mg HBP per one mL of seminal fluid, 0.5% of which was FAA. A purified fraction eluded from RP-HPLC using a 45% acetonitrile buffer at 26 minutes contained only a single HBP band, as indicated by blotting with M1, at 31 kDa. FAA is indicated to bind to sperm cells and is extractable by treatment with hypertonic media (0.6 N Kcl). Analysis of proteins isolated from additional glands, confirmed production in the seminal vesicles and bulbourethral glands, but not in the prostate gland. FAA has a pI of approximately 8.1. FAA does not contain carbohydrate regions. In the N-terminal amino acid sequence provided, X is an undetermined amino acid, and is likely cysteine. The amino acid sequence comparisons set forth in FIG. 1 shows a strong similarity with the compared protein.

HBP proteins bind to spermatozoa at ejaculation, and exposure of sperm to seminal fluid HBP mediates capacitation by heparin. HBP complexes with the greatest affinity for heparin contained the 31 kDa FAA protein, as well as the 24 kDa protein discussed below, which are not present in HBP complexes with the least affinity for heparin. Miller et al., 1990. This suggests that the 31 and 24 kDa HBP regulate high affinity heparin binding to sperm. Sperm from high fertility bulls bind heparin with greater affinity than sperm lower fertility bulls. Marks et al., 1985. Therefore, bull semen samples reflecting higher concentration of FAA, alone, or together with TIMP-2 discussed below, indicate that the donors have higher potential fertility, and offer grounds for selection for subsequent breeding.

### Materials and Methods

### Isolation of Fertility-Associated Antigen (FAA)

Fertility-associated antigen (FAA) was isolated from bovine seminal plasma, sperm membranes, and accessory sex glands. Seminal plasma used for protein purification was collected by an artificial vagina from a vasectomized Holstein bull housed at Sire Power, Inc., Tunkhannock, Pa., frozen in liquid nitrogen, and shipped to the University of Arizona. Seminal plasma was centrifuged at 12,000×g for five min at 4° C. prior to use. Five-hundred μL to 1 mL of seminal fluid supernatant (sperm extracts or accessory sex gland proteins; described below) was injected onto a heparin-affinity column (heparin econo-pac, Bio-Rad, Hercules, Calif.; or an 0.5 cm×25 cm column of heparin-sepharose CL-6B, Pharmacia) connected in-line to an ISCO peristaltic pump operating at a flow rate of approximately 1 mL/min. The column was equilibrated with 40 mM Tris-Cl (ph 7.4), 2 mM CaCl₂, 200 μM PMSF, 0.01% NaN₃ (TC-A). Peak detection was achieved by monitoring absorbance at 280 nm with an UA-5 absorbance detector (ISCO, Inc. Lincoln, Nebr.). After achieving baseline conditions, bound protein (HBP) was eluted with 2M NaCl in TC-A, and fractions (15-mL/fraction) were desalted and concentrated by centrifugation (Beckman J-6M, 2,000×g) in centriprep tubes (Ultrafree-15, 12,000 M.Wt. cut-off, Millipore, Bedford, Mass.). All separations took place at 4° C. Unbound and bound fractions were assayed to determine protein concentration (Bio-Rad Dc protein assay, Bio-Rad, Hercules, Calif.), using BSA as standard, then immediately frozen and lyophilized.

Lyophilized powder was resuspended in buffer A (95% H₂O/5% acetonitrile in 0.1% (wt:vol) trifluoroacetic acid [TFA]). Three mg or more of HBP were injected (100 μl-1 mL) onto a C4 reversed-phase HPLC column (Vydac, Hesperia, Calif.) using an Hitachi HPLC autosampler. Proteins were fractionated with a multi-step linear gradient from 25% buffer A to 100% buffer B (70% acetonitrile in 0.085% (wt:vol) TFA) over 55 minutes, with a total run time of 60 min. Thirty-second fractions (1.5 mL/fraction) were collected using a Foxy fraction collector (ISCO, Inc. Lincoln, Nebr.) linked to a diode array detector and dried under vacuum (Speedvac, Savant Instruments, Farmingdale, N.Y.).

### Extraction of FAA from Sperm

Frozen-thawed ejaculated sperm were washed 3× in PBS (ph 7.4 with protease inhibitors) to remove seminal plasma, resuspended in 5-mL of PBS/0.6 N KCl, and agitated for 60 min on ice. The sperm suspension was centrifuged at 1,000×g for 10 min, supernatant was removed, and the supernatant containing the KCl extract was recentrifuged at 14,000×g for 30 min. Salt-extracted sperm were washed 3× in PBS, solubilized in an equal volume of 2× sample buffer, and boiled prior to gel electrophoresis. An aliquot of non-extracted ejaculated sperm was solubilized in 2× sample buffer after washing in PBS. The sperm KCl extract was concentrated and desalted by centrifugation, assayed for total protein concentration (Bio-Rad D_{c} protein assay), and either solubilized in 2× sample buffer or subjected to protein purification as described above.

### Accessory Sex Gland Preparation

Seminal vesicles, prostate, and bulbourethral glands were obtained from a bull immediately after slaughter. Tissues were rinsed in cold buffer (50 mM Tris-Cl (ph 7.5) 150 mM NaCl, 2 mM EDTA, 0.5 mM DTT) and homogenized on ice in 50 mM Tris-Cl (ph 7.5), 1% glycerol, 0.2 mM EDTA, 0.5 mM DTT, 0.5 mM PMSF. Total homogenates from each gland were centrifuged at 14,000×g for 60 min and supernatants were subjected to protein purification as described above.

### SDS-PAGE and Immunoblots

A portion of each HPLC fraction was analyzed by SDS-PAGE and presence of FAA was confirmed by Western blotting using the monoclonal antibody (M1) as previously described (McCauley et al., 1996). Dried fractions were reconstituted in 1× reducing sample buffer (0.1 M sucrose, 3% SDS, 62.4 mM Tris, 2 mM EDTA) and solubilized by boiling for 5 minutes. One dimensional SDS-PAGE was performed according to the method of Laemmli (1970). Ten microliters of prestained molecular weight markers phosphorylase B (105 kDa), BSA (86 kDa), ovalbumin (56 kDa), carbonic anhydrase (35 kDa), soybean trypsin inhibitor (28.8 kDa), and lysozyme (20.5 kDa; Bio-Rad Laboratories, Hercules, Calif.) were applied to one lane. Samples were loaded onto a 5% stacking acrylamide gel and gels were separated with an 8 cm×10 cm denaturing 13.5% polyacrylamide gel (Mighty Small II, Hoefer Scientific Instruments, San Francisco, Calif.) for approximately 90 min at 20 mA constant current per gel. Proteins were then transferred (1 h at 150 mA constant current) using a semi-dry electroblotter (Millipore Milliblot Graphite Electroblotter I) to a PVDF membrane (Trans-blot, Bio-Rad, Hercules, Calif.; or Immobilon P^{sq}, Millipore) with 10 mM 3-cyclohexylamino-1-propane-sulfonic acid (CAPS) in 10% MeOH as electroblotting buffer. Two identical gels were run in parallel, one was stained with Coomassie blue and the other was used for immunoblotting experiments or for obtaining N-terminal amino acid sequence. For sequencing, the PVDF membrane was stained with Ponceau S (0.2% Ponceau S in 1% acetic acid). Immunoblots were carried out with a monoclonal antibody (M1) to detect fertility-associated antigen (FAA) among the heparin binding proteins. Blotted membranes were blocked with 5% BSA in PBS, 3% Tween (PBS-T) for 60 min at room temperature or overnight at 4° C. Hybridoma culture supernatant diluted 1:10 in PBS-T was added for 90 min. at room temperature, membranes were rinsed 3× with PBS-T, and goat-anti mouse IgG horseradish peroxidase conjuugate (BioSource International; Camarillo, Calif.) diluted 1:4000 was added for 60 minutes. Western blots were developed by enhanced chemical luminescence (ECL: Amersham, Northbrook, Ill.) and exposed on x-ray film (Eastman Kodak, Rochester, N.Y.). Images of stained acrylamide gels and Western blots were captured with AlphaImager Digital Analysis software (Alpha Innotech Corp., San Leandro, Calif.).

### Two-Dimensional Polyacrylamide Gel Electrophoresis (2D-PAGE)

The RP-HPLC fraction containing partially purified FAA, confirmed by Western blotting, was subjected to isoelectric focusing with an SE 220 Tube Gel Adapter Kit (Hoefer Scientific Instruments, San Francisco, Calif.) essentially as described by O'Farrell, (1975). Ampholytes (pH 5-8 and pH 3-10) were mixed to establish the pH gradient. Samples were loaded into the IEF tube gels and electrofocused at 500 V for 2.5 h using 20 mM NaOH and 10 mM H₃PO₄ as electrode solutions. IEF gels were placed on a 5% stacking gel, overlayered with tracking dye, and electrophoresed through a 12% running gel. Prestained molecular weight markers were run in an individual lane cast in the stacking gel. Gels were stained with 0.25% coomassie blue and destained with 10% methanol, 5% acetic acid.

### Peptide Mapping

Fertility-associated antigen (FAA) identified by immunoblotting described above was cut from polyacrylamide gels and electroeluted in 50 mM ammonium bicarbonate, 0.1% SDS buffer at 10 mA for 4 h using a Bio-Rad model 422 Electro-eluter. Alternatively, protein was eluted using GeneCapsule (Geno Technology, Inc., St. Louis, Mo.) according to the manufactureers instructions. The electroeluted protein was dried under vacuum, resuspended in 50 μl H₂O, 450 μl cold acetone containing 1 mM HCL and incubated at −20° C. for 3 h to minimize SDS concentrations prior to enzymatic cleavage. Precipitated FAA was washed 2× with 100 μl cold acetone and air dried. FAA was digested with TPCK-trypsin according to the procedure of Stone and Williams (1993). Twenty-five μl of 8 M urea in 0.4 M NH₄HCO₃, and 5 μl of 45 mM dithiothreitol (DTT) were added to the precipitated FAA and incubated for 15 min at 50° C. After cooling to room temperature, 5 μl of 100 mM iodoacetamide were added and incubated for 15 min. Next, 60 μl of H₂O and 5 μl of TPCK-trypsin (0.1 μg/ml) was added and incubated for 24 h at 37° C. The reaction was stopped by freezing or by direct injection onto a C4 column.

In addition, FAA was prepared for peptide mapping by in situ gel digestion or by cleavage of PVDF bound material. In situ gel digestion was done according to the method described by Stone and Williams (1993). Coomassie blue stained protein was cut from the gel, washed in 95% cold acetone, and dried under vacuum. The gel piece was then washed extensively with 0.1 M NH₄HCO₃ prior to reduction with DTT, alkylation by iodoacetamide, and digestion with TPCK-trypsin. Peptides were extracted from the gel by shaking in 500 μl of 0.1 M NH₄HCO₃, supernatant was removed, and 300 μl of 2 M urea, 0.1 M NH₄HCO₃ were added for 24 h. Combined supernatants were dried, resuspended in H₂O/0.1% TFA, and injected on reversed-phase HPLC. Cleavage of PVDF-bound FAA was accomplished by a method described by Legendre and Matsudaira (1989). Ponceaus S-stained FAA was cleaved by Cyanogen Bromide (Aldrich Chemical Co., Milwaukee, Wis.), and cleavage products were solubilized in elution buffer containing 2% SDS, 1% Triton X-100 in 50 mM Tris (ph 9.2). Glycerol and bromphenol blue were added to final concentrations of 6.25% and 0.01%, respectively. The CNBr cleavage mixture was loaded onto a 16% acrylamide gel, electrophoresed, and transferred to PVDF as described.

Tryptic FAA peptides were fractionated with a narrow-bore (2.1 mm×50 mm) reversed-phase C18 column (Vydac, Hesperia, Calif.). Buffer A was 0.06% TFA/H2O, buffer B was 0.052% TFA/80% acetonitrile. Peptides were injected in a volume of 50 μl and separated with a 105 minute linear gradient (Stone and Williams, 1993): 0-60 min (2-37.5% buffer B), 60-90 min (37.5%-75% B), 90-105 min (75%-98% B), at a flow rate of 200 μl per minute using Hitachi HPLC instruments.

### Glycoprotein Analysis

Protein glycosylation was detected using an immunoblot based glycoprotein detection kit (Immun-Blot, Bio-Rad) according to the procedure recommended by the manufacturer to label carbohydrates on PVDF membranes. Briefly, partially purified FAA was separated by SDS-PAGE and transferred to PVDF membranes as described above. The membrane was washed 3× for 10 min with 50 mL of PBS (ph 7.2) and immersed in 50 mL of 10 mM sodium periodate, 5 mM EDTA, 100 mM sodium acetate solution (ph 5.5) in the dark for 20 min. After washing in PBS, the membrane was placed in 50 mL of 100 mM sodium acetate/5 mM EDTA containing 2 μl of biotinylated hydrazide solution and incubated for 60 min. After washing in Tris-buffered saline (TBS, ph 7.2), the membrane was incubated for 1 h at room temperature or overnight at 4° C. with 50 mL of blocking reagent supplied by the vendor. After washing in TBS, the membrane was immersed in streptavidin alkaline phosphatase conjugate, diluted in TBS, for 1 h. Finally, after washing, the membrane was incubated with Nitro Blue Tetrazolium and 5-bromo-4-chloro-3-indolyl phosphate (NBT-BCIP) substrate to visualize carbohydrate containing samples.

### N-terminal Amino Acid Sequencing

Amino acid analysis and protein sequencing were performed at the University of Arizona Macromolecular Structure Facility. Sequence analysis was performed using an Applied Biosystems 477A Protein/Peptide Sequencer interfaced with a 120A HPLC Analyzer (C-18 PTH column) to determine phenylthiohydantoin (PTH) amino acids. Total amino acid analysis was determined with a dedicated Applied Biosystems Model 420A Amino Acid Analyzer with automatic hydrolysis (vapor phase at 160° C. for 100 min. using 6N Hcl) and pre-column with phenylthiocabamyl-amino acid. Similarity searches were performed using the Blast search server of the National Center for Biotechnology Information at the National Library of Medicine.

### TIMP-2

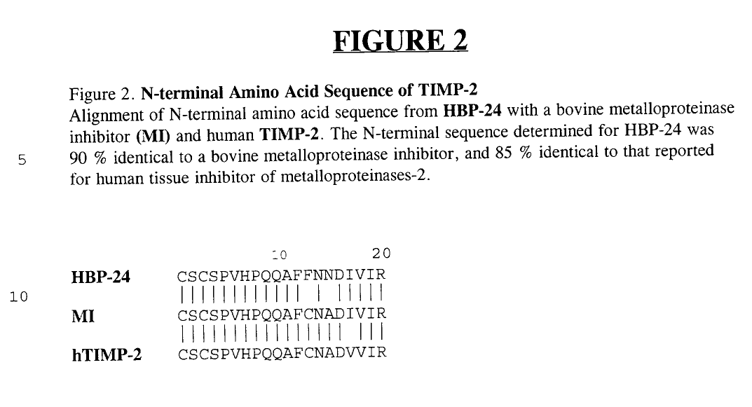

A second fertility-associated protein, having an approximate 24-kDa molecular mass was isolated through methods similar to those used through FAA. Sperm extracts probed with the M1 monoclonal antibody were isolated, and subjected to heparin affinity chromatography and RP-HPLC. A peak eluding in 30% acetonitrile contained an immunoreactive protein that gave a mobility in SDS-PAGE that was greater for the unreduced than the reduced form. Thus, the band migrated at approximately 24-kDa when run in the presence of 2-mercaptoethanol, and migrated at approximately 21.5 kDa under non-reducing conditions. The N-terminal amino acid sequence was obtained from substantially purified material, according to the methods employed in connection with FAA. The N-terminal sequence shared significant homology with a tissue inhibitor of metalloproteinase-2 (TIMP-2), a 21-kDa protein purified from human melanoma cells, and bears close homology with a bovine metalloproteinase inhibitor purified from aortic endothelial cells. DeClerck et al., 1989. This protein was subsequently identified as TIMP-2. Boone et al., 1990. The alignment of these amino acid sequences with HBP-24 is shown in FIG. 2.

Tissue inhibitors of metalloproteinases are a family of multi-functional proteins that form 1:1 stoichiometeric complexes with matrix metalloproteinases, thereby inactivating them. These enzymes have selective activity against many extracellular matrix components. Metalloproteinases are capable of enzymatically degrading virtually all extraceullar matrix constituents. It is, therefore, reasonable, given the high correlation of HBP-24 with fertility to conclude that it is implicated in preventing digestion of sperm surface protein events that are tied to capacitation and zona binding, leading to fertilization.

### MATERIALS AND METHODS

### Reagents

Acetonitrile and trifluoroacetic acid (TFA) were purchased from J. T. Baker, Phillipsburg, N.J.; 30% acrylamide/Bis was from Bio-Rad Laboratories, Hercules, Calif.; TEMED, Ammonium Persulfate, 2-Mercaptoethanol, 3-cyclohexylamino-1-propane-sulfonic acid (CAPS), and other buffer reagents were obtained from Sigma Chemical Company, St. Louis, Mo.

### Isolation of TIMP-2

Seminal fluid from a vasectomized bull (five-hundred μL to one mL) was injected onto a heparin-affinity column (heparin-sepharose CL-6B, Pharmacia) equilibrated with 40 mM Tris-Cl, 2 mM CaCl₂, 200 μM PMSF, 0.01% NaN₃ (TC-A). Absorbance was monitored at 280 nm (UA-5 absorbance detector, ISCO, Inc. Lincoln, Nebr.). The unbound effluent was collected and after achieving baseline conditions, bound protein was eluted with a linear gradient to 2M NaCl. Heparin-binding proteins (HBP) were desalted and concentrated by centrifugation at 2000×g (Ultrafree-15; Millipore, Bedford, Mass.). All separations took place at 4° C. Protein concentrations were determined with the Bio-Rad Dc protein assay (Bio-Rad laboratories, Hercules, Calif.) using BSA as standard. Samples were immediately frozen and lyophilized. Lyophilized powder was reconstituted in 5% acetonitrile in 0.1% (wt:vol) trifluoroacetic acid (TFA). Up to 10 mg of HBP were injected onto a C4 reversed-phase HPLC column (Vydac, Hesperia, Calif.) and separated with a linear gradient from 5% to 70% acetonitrile in 0.1% (wt:vol) TFA in 40 min. using Hitachi HPLC instruments. Thirty-second fractions (1.5 mL) were collected using an automated fraction collector (ISCO, Inc. Lincoln, Nebr.) linked to a diode array detector and dried with a Speedvac (Savant Instruments, Farmingdale N.Y.).

Each HPLC fraction was analyzed by SDS-PAGE and Western blotting using a monoclonal antibody as previously described (McCauley et al., 1996). SDS-PAGE was performed with the Mighty Small II (Hoefer Scientific Instruments, San Francisco, Calif.) under both reducing and non-reducing conditions. Proteins were dissolved in SDS-Laemmli buffer (Laemmli, 1970) and boiled for 5 minutes. Fifty micrograms of protein were loaded in each lane and separated with a 13.5% gel. Following electrophoresis, proteins were in CAPS (10 mM in 10% methanol) buffer for 1 h at 100 mA to a PVDF membrane using a semi-dry blotter (Hoefer, Scientific Instruments; San Francisco, Calif.). Blotted membranes were blocked with 5% BSA in PBS, 3% Tween (PBS-T) overnight at 4° C. Hybridoma culture supernatant was added for 90 min., membranes were rinsed 3× with PBS-T, and goat-anti mouse IgG horseradish peroxidase conjuugate (BioSource International; Camarillo, Calif.) diluted 1:4000 was added for 60 minutes. Western blots were developed by enhanced chemical luminescence (ECL; Amersham, Northbrook, Ill.) and exposed on x-ray film (Eastman Kodak, Rochester, N.Y.). Images of stained acrylamide gels and Western blots were captured with Alphalmager Digital Analysis software (Alpha Innotech Corp., San Leandro, Calif.).

N-terminal amino acid sequencing was performed at the University of Arizona Macromolecular Structure Facility. Sequence analysis was performed using an Applied Biosystems 477A Protein/Peptide Sequencer interfaced with a 120A HPLC Analyzer (C-18 PTH column) to determine phenylthiohydantoin (PTH) amino acids. Total amino acid analysis was determined with a dedicated Applied Biosystems Model 420A Amino Acid Analyzer with automatic hydrolysis (vapor phase at 160° C. for 100 min. using 6N Hcl) and pre-column with phenylthiocabamyl-amino acid. Similarity searches were performed using the Blast program.

### Applications

Both FAA and TIMP-2 are highly correlated with fertility. FAA is correlated with a 19% difference increase in average fertility, while TIMP-2 is correlated with a 16% difference. Thus, immunoassays for the presence of either FAA or TIMP-2, by HBP selected monoclonal antibodies, or by RP-HPLC, provide a convenient and automatable laboratory assay for determining which bulls of a group may be likely to be more fertile. These proteins are secreted by accessory glands and bind to sperm as they traverse the male reproductive tract. Assays, therefore, must be made on seminal secretions, and not based on sperm leaving the testis. Assays searching for the presence of both FAA and TIMP-2 give a more reliable indication of potential fertility.

The proteins of this invention, as well as methods of purifying the same and assaying for the same have been described both generically, and by reference to specific embodiments. These embodiments are not limiting, and alternatives to those of ordinary skill in the art, particularly in terms of assay format and the like, will occur without the exercise of inventive faculty. Such alternatives remain within the scope of the invention, unless delimited by the recitation of the claims set forth below.

### References

Bellin, M E, H E Hawkins, and R L Ax. Fertility of range beef bulls grouped according to presence or absence of heparin-binding proteins in sperm membranes and seminal fluid. J Anim Sci 1994; 72:2441-2448.

Bellin M E, Hawkins H E, Oyarzo J N, Vanderboom R J, and R L Ax. Monoclonal antibody detection of heparin-binding proteins on sperm corresponds to increased fertility of bulls. J Anim Sci 1996; 74:173-182.

Bellin M E, Oyarzo J N, Hawkins H E, Zhang H, Smith R G, Forrest D W, Sprott L R, and R L Ax. Fertility associated antigen on bull sperm indicates fertility potential. J Anim Sci 1998; In Press.

Boone T C, Johnson M J, DeClerck Y A, and K E Langley. CDNA cloning and expression of a metalloproteinase inhibitor related to tissue inhibitor of metalloproteinases. Biochem 1990; 87:2800-2804.

Cancel, A M, Chapman D A, and G J Killian. Osteopontin is the 55-kilodalton fertility-associated protein in holstein bulls seminal plasma. Biol Reprod 1997; 57:1293-1301.

Chenoweth P J, Spitzer J C, and F M Hopkins. A new bull breeding soundness evaluation form. Proc Ann Mtng Soc for Therio, San Antonio, Tex., 1992.

DeClerck Y A, Yean T D, Ratzkin B J, Lu H S, and K E Langley. Purification and characterization of two related but distinct metalloproteinase inhibitors secreted by bovine aortic endothelial cells. J Biol Chem 1989; 264:17445-17453.

Gerena R L, Irakura D, Urade Y, Eguchi N, Chapman D A, and G J Killian. Identification of a fertility-associated protein in bull seminal plasma as lipocalin-type prostaglandin D synthase. Biol Reprod 1998; 58:826-833.

Graham J K, Kunze E, and R H Hammerstedt. Analysis of sperm viability, acrosomal integrity, and mitochondrial function using flow cytometry. Biol Reprod 1990; 43:55-64.

Killian G J, Chapman D A, and L A Rogowski. Fertility-associated proteins in Holstein bull seminal plasma. Bio Reprod 1993; 49:1202-1207.

LeGendre N, and P Matsudaira. Purification of Proteins and Peptides by SDS-PAGE. In: A Practical Guide to Protein and Peptide Purification for Microsequencing. P Matsudaira (ed.). 1989; Academic Press, San Diego.

Lenz R W, Martin J L, Bellin M E, and R L Ax. Predicting fertility of dairy bulls by inducing acrosome reactions in sperm with chondroitin sulfate. J Dairy Sci 1988; 71:1073-1077.

Marks J L, and R L Ax. Relationship of nonreturn rates of dairy bulls to binding affinity of heparin to sperm. J Dairy Sci 1985; 68:2078-2082.

Miller D J, Winer M A, and R L Ax. Heparin-binding proteins from seminal plasma bind to bovine spermatozoa and modulate capacitation by heparin. Biol Reprod 1990; 42:899-915.

O'Connor M T, Amann R P, and R G Saacke. Comparisons of computer evaluations of spermatozoal motility with standard laboratory tests and their use for predicting fertility. J Anim Sci 1981; 53:1368-1376.

Rodriguez A M, Rodin D, Nomura H, Morton C C, Weremowicz S, and M C Schneider. Identification, localization, and expression of two novel human genes similar to deoxyribonuclease I. Genomics 1997; 42:507-513.

Saacke R G, and J M White. Semen quality tests and their relationship to fertility. Proc. 4^{th} Tech Conf Artif Insem Reprod., Natl Assoc Anim Breeders 1972; p 2-7.

Saacke R G. Semen quality in relation to semen preservation. J Dairy Sci 1983; 66:2635-2644.

Saling P M. Mammalian sperm interaction with extracellular matrices of the egg. S R Milligan (Ed.) In: Oxford reviews of reproductive biology. 1989; 339-388. Oxford University Press, New York.

Stone K L, and K R Williams. Enzymatic digestion of proteins and hplc peptide isolation. In: a practical guide to protein and peptide purification for microsequencing. P Matsudaira (ed.), 1993: Academic Press, San Diego.

Trenkle A. and R. L. Wilham. Beef Production Efficiency. Science 1977; 198:1009.

Whitfield C H, and T J Parkinson. Relationship between fertility of bovine semen and in vitro induction of acrosome reactions by heparin. Therio 1992; 38:11.

What is claimed is:

1. A substantially purified protein which is correlated with enhanced fertility in mammalian males, wherein said protein is bound by monoclonal antibody M1, and is selected from the group consisting of (1) a non-glycosylated protein migrating at 31 kDa in one dimensional SDS-PAGE electrophoresis and (2) a tissue inhibitor of metalloproteinase exhibiting a 24 kDa migration in one-dimensional SDS-PAGE electrophoresis in reduced form, and a 21.5-kDa molecular mass under non-reducing conditions, wherein said substantially purified proteins are secreted by seminal vesicles.

2. The substantially purified protein of claim 1, wherein said protein is said 31-kDa protein, is a heparin binding protein, and has an isoelectric point of approximately 8.3.

3. The protein of claim 2, wherein said substantially purified protein has an N-terminal sequence LKIXSFNVRSFGESKKAGFNAMRVIV and, when subject to lys-C digestion, results in fragments having the amino acid sequences QSYLYHDYQAGDADVFSREP and DFVIVPLHTTPEXXV, wherein X indicates any amino acid.

4. The substantially purified protein of claim 1, wherein said protein exhibits a 24-kDa molecular mass in SDS-PAGE electrophoresis in the presence of 2-mercaptoethanol, and has an N-terminal sequence CSCSPVHPQQAFFNNDIVIR.

5. The substantially purified protein of claim 1, wherein said mammalian male is a bull.

6. A method of identifying enhanced fertility in mammalian male out of a group of such males, comprising assaying semen samples from each member of said group to determine the presence in said semen sample of the protein of claim 1, wherein the presence of said protein in a semen sample is indicative of higher fertility in the donor of said semen sample.

7. The method of claim 6, wherein said mammalian male is a bull.

8. The method of claim 7, wherein said assay is conducted using an immunoassay wherein said semen samples are combined with a monoclonal antibody specific for said protein, and binding between said monoclonal antibody and protein in said sample is indicative of higher fertility.

9. The method of claim 8, wherein the amount of binding activity occuring is quantified, and wherein a higher degree of binding is indicative of greater fertility.
